(19) United States
(12) Patent Application Publication
(10) Pub. No.: US 2008/0146816 A1
(43) Pub. Date: Jun. 19, 2008

(54) MONOMER OF THE "PUSH-PULL" TYPE AND PHOTOCHROMIC ELECTROCONDUCTING POLYMER MATERIAL OBTAINED FROM THIS MONOMER

(75) Inventors: Isabelle Fabre-Francke, Grenoble (FR); Said Sadki, St. Martin d'Heres (FR)

Correspondence Address: OBLON, SPIVAK, MCCLELLAND MAIER & NEUSTADT, P.C., 1940 DUKE STREET, ALEXANDRIA, VA, 22314, US

(73) Assignee: COMMISSARIAT A L'ENERGIE ATOMIQUE, Paris (FR)

(21) Appl. No.: 11/612,169

(22) Filed: Dec. 18, 2006

(51) Int. Cl. C07D 209/82; C07D 233/00

(52) U.S. Cl. 548/335.1; 548/440

(57) ABSTRACT

The invention relates to a “push-pull” type compound, used for manufacturing polymer materials having both electrical conduction and photochromic properties, which responds to the general formula A-X-R in which:
A is an electron acceptor group;
X is a group having a π-conjugated system and forms a photochromic group with A; and
R is a polymerizable electron-donor group that is chosen among the carbazole group and the groups derived from the carbazole group by substitution, and which is linked to X by the nitrogen atom of the carbazole group.

The invention also relates to an electroconducting and photochromic polymer material, obtained by polymerization of this compound.

Applications: optoelectronics, signal processing, data storage, etc.

### TECHNICAL FIELD

The present invention relates to a “push-pull” type compound, used for manufacturing polymers having both electrical conduction properties and photochromic properties, that is to say an ability to change colour, reversibly, under the effect of light radiation.

It also relates to an electroconducting and photochromic polymer material, which is obtained by polymerization of this compound.

Such a material is especially likely to find applications in optoelectronics but also in the field of signal processing and data storage for telecommunications, imaging (for example, medical imaging), audio and video, etc.

### STATE OF THE PRIOR ART

During recent years, a particular interest has been taken in electroconducting polymers owing to the very many potential applications that polymers of this type have.

In particular, numerous studies have been devoted to the development of polymers possessing, besides an ability to conduct electricity, electrochromic, photochromic or nonlinear optical properties with a view to applications in optoelectronics or for signal processing and data storage.

There are essentially two methods for conferring photochromic properties on a polymer.

The first, which is described, for example by Inaba et al. in Macromolecules, 1996, 29, 2954-2959, [1], consists in inserting a photochromic compound within a polymer matrix. In this case, there is no covalent bond between the photochrome and the polymer. Besides the fact that the materials thus obtained may have morphological defects and insufficient mechanical properties, demixing problems may occur on account of the high segregation power that the photochromic compounds generally have. In addition, it is difficult to produce in that way materials having a high photochrome content.

The second consists in grafting a chromophore onto the polymer by chemical reaction. Therefore, in this case it is a question of chemically modifying the polymer. The major drawback of this method lies in the fact that it is difficult to control the degree of grafting of the chromophore group and therefore to guarantee reproducibility of the properties of the materials thus obtained. In addition, to the knowledge of the inventors, it has never been applied to electroconducting polymers and to date has only been used for polymethacrylate, polyamide, polyimide or polyester type polymers for applications in nonlinear optics.

In addition, Thompson et al. have described in New J. Chem., 2005, 29, 1128-1134, [2], the preparation of an electroconducting polymer material by electropolymerization of a monomer composed of a photochromic group, namely a salicylidene aniline group, linked covalently to a polymerizable group, such as a 2,5-dithienylpyrrole group. This material was shown however to have no photochromic properties.

The inventors were therefore set the objective of providing polymer materials that have both electrical conduction and photochromic properties and of which the photochromy is obtained neither by inserting a photochrome into these materials, nor by chemical modification of the polymer constituting them.

In addition, they were set the objective that the preparation of these materials be simple to implement and have a cost compatible with an industrial working.

### SUMMARY OF THE INVENTION

These objectives and others are achieved by the invention that proposes, in the first place, a compound able to be polymerized, especially by an electrochemical route, and of which the polymerization results in the production of electroconducting and photochromic polymer materials.

This compound, which is of the “push-pull” type, responds to the general formula (I) below:

A-X-R (I)

in which:
A represents an electron-acceptor group;
X represents a group having a π-conjugated system and forms a photochromic group with A; while
R represents a polymerizable electron-donor group that is chosen among the carbazole group and the groups derived from the carbazole group by substitutions, and which is linked to X by the nitrogen atom of the carbazole group.

In the preceding and following text, the term “photochromic group” is understood to mean a group having the property of changing colour under the effects of a light radiation (visible, infrared or ultraviolet light), then of returning to its original colour when the light radiation stops or via excitation, while the term “polymerizable group” is understood to mean a group capable of reacting with itself to form a polymer.

According to the invention, the photochromic group formed by A and X may be chosen among the very many groups known for having photochromic properties in so far as these groups be made up of an electron-acceptor group linked covalently to a group having a π-conjugated system.

Thus, this photochromic group may especially be chosen among anil, diarylethene, hexaarylbiimidazole, spiropyran, azobenzene and norbornadiene groups.

In this regard, the reader will find in the article “Organic Photochromism” by Bouas-Laurent and Dürr (Pure Appl. Chem., 73 4, 639-665, [3]) and in the work “Organic Photochromic and Thermochromic compounds: Volume 1: Photochromic Families (Topics in Applied Chemistry)” edited by John C. Crano and Robert J. Guglielmi (1999, Springer) a description of the main types of photochromic molecules known to date, their structure and their properties.

As mentioned previously, the polymerizable group R may itself be a carbazole group or else a group derived from this by substitution.

Although, in the latter case very many types of substitutions may be envisaged, it is preferred that the substituent(s) of the carbazole group be chosen among groups that comprise one or more double bonds forming a π-conjugated system with said carbazole group in order to promote the transfer of electrons between the carbazole group and this or these substituents and, in the same way, the electroconducting properties of the polymer materials.

These unsaturated groups may be aliphatic groups (linear or branched), cyclic groups (mono- or polycyclic) and also partially aliphatic and partially cyclic groups. In addition, they may comprise one or more heteroatoms (nitrogen, oxygen and sulphur especially) or be exclusively hydrocarbon-based. That said, it is more particularly preferred that the polymerizable group R be a carbazole group substituted by two cyclic or heterocyclic groups that comprise several double bonds forming a π-conjugated system with said carbazole group. In which case, the first of these cyclic or heterocyclic groups is advantageously borne by the carbon atom located at position 3 of the carbazole group, while the second of these cyclic or heterocyclic groups is preferably borne by the carbon atom located at position 6 of this same group, said positions being defined as follows:

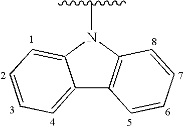

Thus, for example the polymerizable groups R being shown to be particularly well suited are:
carbazole groups substituted by two 3,4-ethylenedioxythiophene (EDOT) groups such as, for example the EDOT-carbazole-EDOT group of formula (II) below:

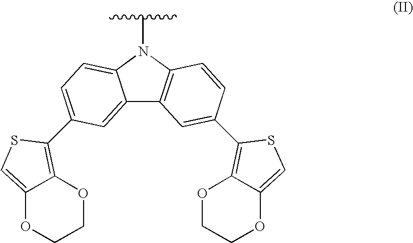

carbazole groups substituted by two 2,3-dihydro-5-(2,3-dihydrothieno[3,4][1,4]-dioxin-5-yl)thieno-[3,4][1,4]dioxin groups (bisEDOT) groups such as, for example the bisEDOT-carbazole-bisEDOT group of formula (III) below:

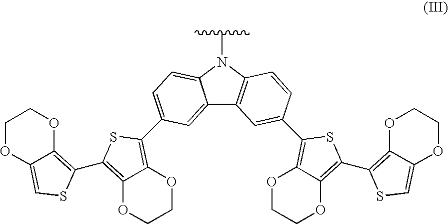

and
carbazole groups substituted by two azulene groups such as, for example the azulene-carbazole-azulene group of formula (IV) below:

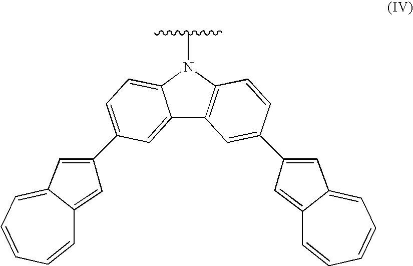

Indeed, it turns out that the presence on the carbazole group of EDOT, bisEDOT or azulene type groups, whose oxidation potential is less than 1 V, makes it possible to reduce the oxidation potential of the carbazole group and thus to confer on the compound according to the invention, when it is polymerized by an electrochemical route, an overoxidation resistance greater than that which it would have in the absence of these groups. In addition, it increases the molecular weight of the compound according to the invention and, as a consequence, its degree of polymerization. The polymers are therefore made up of longer chains, which improves their mechanical properties. Lastly, it has the advantage of resulting in the formation of linear polymers that are generally more stable than branched polymers.

As a variant, it is however also possible that the compound according to the invention comprises as the polymerizable group R, a carbazole group substituted by two 3,4-ethylenedioxypyrrole (EDOP) groups or by two 3,4-(1,3-propylenedioxy)pyrrole (PropDOP) groups or else by two 3,4-(1,3-butylenedioxy)pyrrole (BuDOP) groups, these groups being, here as well, preferably borne by the carbon atoms located at positions 3 and 6 of the carbazole group.

In a preferred embodiment of the compound according to the invention, the photochromic group formed by A and X is chosen from anil groups.

Anils, which are also called salicylidene anilines as they result from the condensation of a salicylaldehyde derivative with an aniline derivative in an alcohol solution, are known for having thermochromic and photochromic properties in the solid state. These properties appear to be linked to an intramolecular proton transfer being characterized by the induction of keto-enol tautomerism (conversion from an enol form to a ketone form) under certain conditions (irradiation at about 400 nm). Moreover there is a cis-trans isomerization equilibrium of the ketone form which could be the origin of the difference between thermochromism and photochromism: cis-ketone in the case of thermochromism and trans-ketone in the case of photochromism. Anils have a type T photochromism, that is to say that the return to the enol form may be induced by heat or by an excitation at around 500 nm. This return takes place with a relaxation constant of a few milliseconds in solution and which may vary from a few seconds to a few hundred days in the solid state. They are therefore of particular interest in nonlinear optics for data storage type applications (by varying the relaxation times) or for optical switches.

According to the invention, the anil group formed by A and X preferably responds to the formula (V) below:

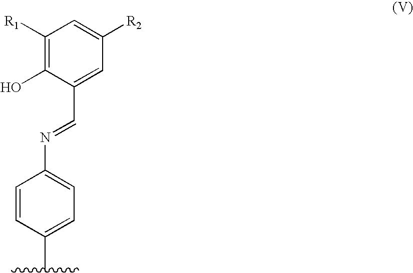

in which R₁ and R₂, which are preferentially but not necessarily identical, represent a halogen atom (chlorine, bromine, iodine or fluorine), a nitro group or else a linear or branched alkyl or alkoxy group comprising from 1 to 6 carbon atoms and preferably from 1 to 4 carbon atoms. Better yet, R₁ and R₂ represent a tert-butyl group.

When the photochromic formed by A and X is an anil group, then it fits that the polymerizable group R has an oxidation potential less than the oxidation potential of this anil group so as to prevent, during the polymerization, if this is carried out by an electrochemical route, the phenol group from being oxidized before the polymerizable group R and from being transformed in this way into a quinone, which has the effect of irreversibly destroying the photochromic properties of the anil group.

The polymerizable group R is therefore, in this case, preferably a carbazole group substituted by two EDOT, bisEDOT or azulene groups, such as those represented hereinabove.

A particularly preferred compound according to the invention responds to the formula (VI) below:

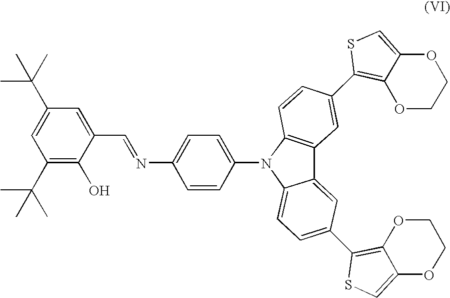

The compound according to the invention may be prepared by synthetic pathways within the scope of a person skilled in the art.

The starting compound is generally carbazole that is subjected to one or more successive coupling reactions in order to graft the photochromic group onto the pyrrole ring of the carbazole. When the polymerizable group R is a group derived from carbazole by substitution, then the product obtained is in turn subjected to one or more coupling reactions in order to derivatize said carbazole group.

These coupling reactions such as, for example Stille coupling or Suzuki coupling, which in particular allow substituents to be grafted onto the benzene rings and which are both catalysed by palladium, are commonly used reactions in organic synthesis. It is also possible to use, for this type of coupling, the reaction known as the Grignard reaction.

Another subject of the invention is an electroconducting and photochromic polymer material, which is obtained by polymerization of a compound as defined previously.

This polymer material is preferably in the form of a film, of which the thickness may range from a few nanometers to a few tens of micrometers depending on the application for which it is intended.

Such a film may be obtained by polymerizing the compound according to the invention via a chemical route, then by depositing the resultant polymer onto a support, for example by spin coating or dip coating.

However, with regard to the applications to which the polymer material is intended, it is preferred to turn to a polymerization via an electrochemical route, that is to say to an electropolymerization, since it makes it possible to form the polymer directly on the surface of a conductive support such as, for example an ITO (indium tin oxide) support and to control the thickness of the film which is formed by coulometry or profilometry.

The invention will be better understood in the light of the remainder of the description, which refers to examples of synthesizing a compound according to the invention and a polymer film having this compound as a monomer.

Of course, these examples are only given by way of illustration of the invention and are in no way limiting.

### BRIEF DESCRIPTION OF THE DRAWINGS

FIG. 1 represents the outline of the synthesis of a compound according to the invention.

FIG. 2 represents the voltammogram obtained during the electropolymerization by cyclic voltammetry of the compound whose synthesis is illustrated in FIG. 1.

FIG. 3 represents the voltammogram obtained during a cyclic voltammetry analysis aimed at characterizing a polymer film prepared by electropolymerization of the compound whose synthesis is illustrated in FIG. 1.

FIG. 4 represents the voltammogram obtained during a cyclic voltammetry analysis aimed at assessing the stability of a polymer film prepared by electropolymerization of the compound whose synthesis is illustrated in FIG. 1.

### DETAILED SUMMARY OF EMBODIMENT EXAMPLES

### Example 1

### Synthesis of a Compound According to the Invention

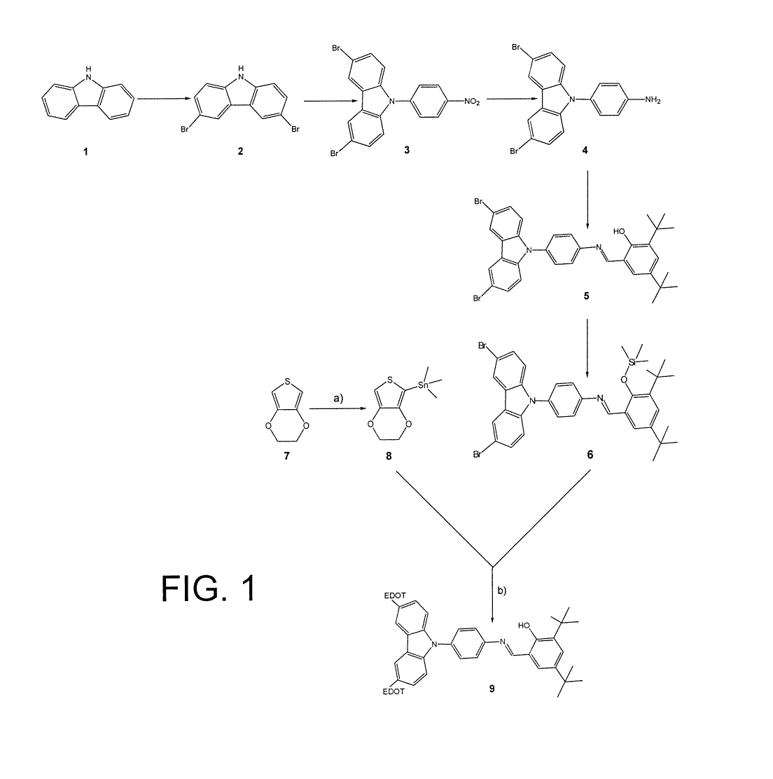

The compound responding to formula (VI) above, which was referenced 9 on FIG. 1, was synthesized according to the reaction scheme illustrated on this figure from two commercially available compounds: carbazole or compound 1 on the one hand, and 3,4-ethylenedioxy-thiophene or compound 7 on the other hand.

### Synthesis of Compound 2:

Compound 2 was obtained by dibromination of compound 1.

This dibromination was carried out by making this compound (2.01 g, 12.0 mmol) react with N-succinimide (4.20 g, 23.8 mmol), in the presence of silica preactivated at 120° C. (40.50 g, 0.063-0.2 nm) in order to protonate the nitrogen atom of compound 1, and in dichloromethane (350 ml), as described by Smith et al. in Tetrahedron, 1992, 48, 36, 7479-7488, [4].

The yield of the reaction was 76%.

### Synthesis of Compound 3:

Compound 3 was obtained by coupling compound 2 with 4-fluoronitrobenzene.

This coupling was carried out by making the compound 2 (1.00 g, 3.1 mmol), previously dissolved in dimethylformamide (15 ml), react with 4-fluoronitrobenzene (699.40 mg, 4.9 mmol) in the presence of sodium hydride (236.00 mg, 9.8 mmol) in order to deprotonate the nitrogen atom of compound 2, as described by Zhu et al. in Macromolecules, 2000, 33, 801-807, [5].

However, unlike the protocol proposed by Zhu et al., the solvents (CH₂Cl₂ and hexane) used for washing the precipitate formed were at 0° C.

The yield of this reaction was quantitative.

### Synthesis of Compound 4:

Compound 4 was obtained by reduction of the nitro group of compound 3 to an amine group.

This reduction was carried out according to the protocol described by Davey et al. in Chem. Mater., 2000, 12, 1679-1693, [6].

In order to do this, a solution of sodium sulphide nonahydrate (2.20 g, 9.1 mmol) and sulphur (307.50 mg, 9.6 mmol) in water (5 ml) was added to compound 3 (1.20 g, 2.7 mmol), previously dissolved under heat in pyridine (10 ml), then the reaction medium was put under reflux. It was then dissolved with water (15 ml) and it was left under stirring, under argon and at room temperature, for one night. The precipitate thus formed was filtered then washed with water.

The yield of the reaction was 98%.

### Synthesis of Compound 5:

Compound 5 was obtained by condensation of compound 4 (467.40 mg, 1.1 mmol), previously dissolved in ethanol (30 ml), with 3,5-di-tert-butyl-2-hydroxybenzaldehyde (290.10 mg, 1.2 mmol), as described by Chong et al. in Org. Lett., 2003, 5, 21, 3823-3826, [7].

The yield of this reaction was 79%.

### Synthesis of Compound 6:

Compound 6 was obtained by silylation of compound 5 (1.00 g, 1.6 mmol) with chloromethylsilane (1.4 ml, 11.0 mmol).

This silylation, which has no other objective than that to protect the phenol functional group of compound 5 with a tert-silyl group before proceeding with the Stille coupling necessary for obtaining compound 7, was carried out at room temperature, in dichloromethane (25 ml) and in the presence of diethylamine (1.30 ml, 9.3 mmol) and 4-dimethylaminopyridine (1.11 g, 9.1 mmol) as a catalyst.

### Synthesis of Compound 9:

Compound 9 was obtained in two substeps:
a substep a) which consisted in making compound 7 (EDOT) react with trimethyltin chloride (ClSnMe₃) in order to obtain compound 8 according to the protocol described by Edder et al. in Org. Lett., 2003, 5, 1879-1882, [8]; then
a substep b) which consisted in coupling, by Stille coupling, compounds 6 and 8 in order to obtain compound 9.

In substep a), compound 7 (710 μl, 6.7 mmol) was dissolved in tetrahydrofuran (14 ml) then, at −78° C., 2.5 M n-butyllithium in pentane (5.2 ml, 1.5 mmol) was slowly added to the solution thus obtained and the temperature of the mixture rose back up to room temperature. This mixture was returned to −78° C. and ClSnMe₃ (1.38 g, 1.0 mmol) was added to it. The reaction medium was left under stirring, at room temperature and under argon, for one night. Then it was diluted with diethyl ether, washed with ammonium chloride, dried over sodium sulphate, filtered and evaporated. Compound 8 was thus obtained with a yield of 72%.

In substep b), compound 6 (1.1 g, 1.6 mmol), was dissolved in tetrahydrofuran (20 ml), then at room temperature tetrakis(triphenylphosphine)palladium (Pd(PPh₃)₄) (catalyst) (131.4 mg, 0.1 mmol) and compound 7 (1.2 g, 4.0 mmol) were added. The reaction medium was placed, under stirring and under argon, under reflux in tetrahydrofuran for 24 hours. Then it was diluted in diethyl ether, washed with ammonium chloride, dried over sodium sulphate, filtered and evaporated. Thereafter, it was purified on a silica column (particle size: 0.063-0.2 nm) with an eluent ranging from a 4:1 v:v hexane/dichloromethane mixture to pure dichloromethane.

Compound 9 was thus obtained in the form of a yellow solid, with a yield of 40%.

### Example 2

### Synthesis of a Polymer Film According to the Invention

A polymer film according to the invention was prepared by electropolymerization of compound 9 as obtained in Example 1 above.

This electropolymerization was carried out by cyclic voltammetry, in a medium comprising the compound 9 at a concentration of 1.0 mmol/l, tetrabutylammonium-perchlorate as the electrolyte, at a concentration of 0.1 mol/l, and acetonitrile as the solvent.

The working electrode and the counter electrode were made of platinum, while the reference electrode was a Ag/AgCl electrode.

The operating parameters were the following ones:
reaction potential: 0.05-0.96 V
sweep rate: 0.05 V/s
number of scans: 10.

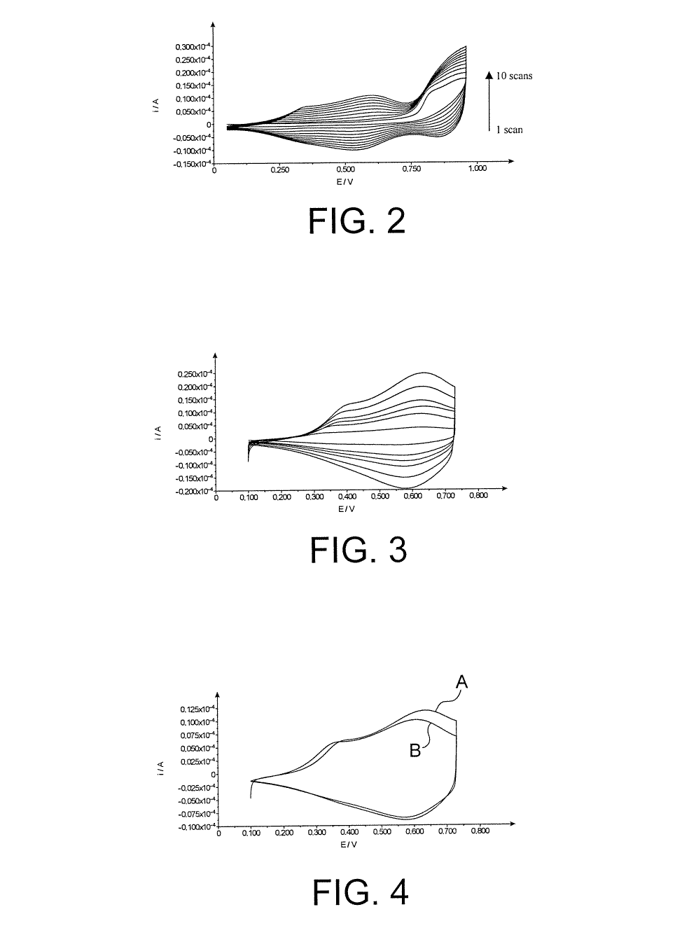

In FIG. 2, which corresponds to the voltammogram obtained during this electropolymerization, it is possible to see the growth of the polymer and the formation of new waves that are characteristic of this.

Thus the formation of a coloured (yellow/green) polymer film with a thickness of around 100 nm on the platinum working electrode was obtained.

This polymer film was analysed by cyclic voltammetry under the following conditions:
medium: 0.1 M NBu₄ClO₄/acetonitrile;
working electrode and counter electrode: Pt;
reference electrode: Ag/AgCl;
reaction potential: 0.1-0.73 V;
sweep rate: 0.02-0.1 V/s; and
number of scans: 1.

The voltammogram thus obtained, which is illustrated in FIG. 3, shows that the polymer is electroactive, that is to say it conducts electricity in the oxidized state. In addition it has a behaviour known as super-capacitor. It is also possible to see on this figure the radical cation form (Eₚ⁺=0.35 V) and the dication form (E_{p1/2}⁺⁺=0.614 V) that are characteristic of this type of polymer.

Furthermore, the stability of the polymer film is assessed by submitting it to 50 scans (sweep rate: 0.05 V/s) and comparing its electroactivity to that observed after a single scan.

As is shown in FIG. 4, the electroactivity of the polymer film is, after 50 scans (curve B) slightly less than that observed after a single scan (curve A), but the difference is small enough for the polymer to be considered stable.

### Documents Cited

[1] Inaba et al., Macromolecules, 1996, 29, 2954-2959.
[2] Thompson et al., New J. Chem., 2005, 29, 1128-1134.
[3] Bouas-Laurant and Dürr, Pure Appl. Chem., 73, 4, 639-665.
[4] Smith et al., Tetrahedron, 1992, 48, 36, 7479-7488.
[5] Zhu et al., Macromolecules, 2000, 33, 801-807.
[6] Davey et al., Chem. Mater., 2000, 12, 1679-1693.
[7] Chong et al., Org. Lett., 2003, 5, 21, 3823-3826.
[8] Edder et al., Org. Lett., 2003, 5, 11, 1879-1882.

1. “Push-pull” type compound, that responds to the general formula (I) below:
A-X-R (I)
in which:
A represents an electron-acceptor group;
X represents a group having a π-conjugated system and forms a photochromic group with A; while
R represents a polymerizable electron-donor group that is chosen among the carbazole group and the groups derived from the carbazole group by substitutions, and which is linked to X by the nitrogen atom of the carbazole group.

2. Compound according to claim 1, in which the photochromic group formed by A and X is chosen among the anil, diarylethene, hexaarylbiimidazole, spiropyran, azobenzene and norbornadiene groups.

3. Compound according to claim 1, in which the polymerizable group R is a carbazole group that is substituted by one or more groups that comprise one or more double bonds forming a π-conjugated system with said benzene ring.

4. Compound according to claim 3, in which the polymerizable group R is a carbazole group substituted by two cyclic or heterocyclic groups that comprise several double bonds forming a π-conjugated system with said carbazole group.

5. Compound according to claim 4, in which one of the two cyclic or heterocyclic groups is borne by the carbon atom located at position 3 of the carbazole group, while the other is borne by the carbon atom located at position 6 of this same group, said positions being defined as follows:

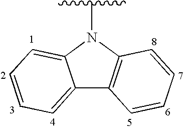

6. Compound according to claim 4, in which the polymerizable group R is a carbazole group substituted by two 3,4-ethylenedioxythiophene groups.

7. Compound according to claim 4, in which the polymerizable group R is a carbazole group substituted by two 2,3-dihydro-5-(2,3-dihydrothieno[3,4][1,4]-dioxin-5-yl)thieno[3,4][1,4]dioxin groups.

8. Compound according to claim 4, in which the polymerizable group R is a carbazole group substituted by two azulene groups.

9. Compound according to claim 4, in which the polymerizable group R responds to any one of the formulae (II), (III) or (IV) below:

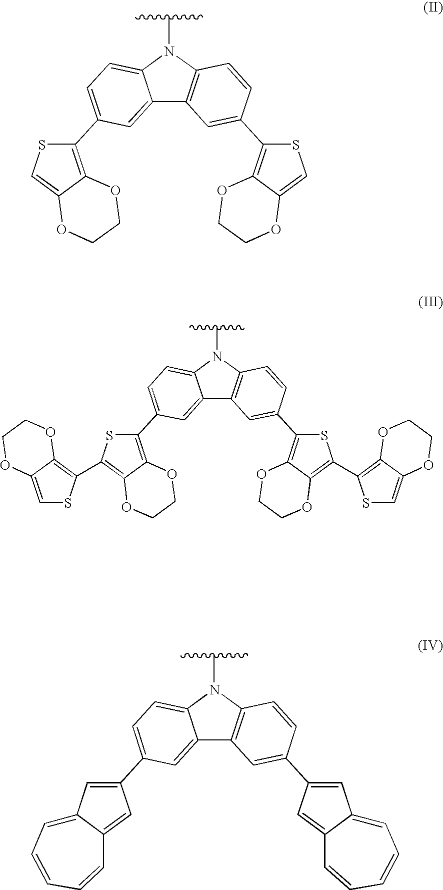

10. Compound according to claim 1, in which the photochromic group is an anil group.

11. Compound according to claim 10, in which the anil group responds to the formula (V) below: 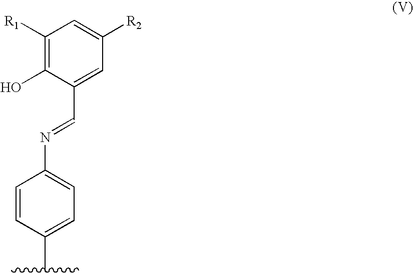
in which R₁ and R₂ represent a halogen atom (chlorine, bromine, iodine or fluorine), a nitro group, or else an alkyl or alkoxy group comprising from 1 to 6 carbon atoms.

12. Compound according to claim 11, in which R₁ and R₂ represent an alkyl or alkoxy group comprising from 1 to 4 carbon atoms.

13. Compound according to claim 12, in which R₁ and R₂ represent a tert-butyl group.

14. Compound according to claim 10, in which the polymerizable group R is a carbazole group substituted by two 3,4-ethylenedioxythiophene groups.

15. Compound according to claim 10, in which the polymerizable group R is a carbazole group substituted by two 2,3,dihydro-5-(2,3-dihydrothieno[3,4][1,4]-dioxin-5-yl)thieno[3,4][1,4]dioxin.

16. Compound according to claim 10, in which the polymerizable group R is a carbazole group substituted by two azulene groups.

17. Compound according to claim 10, in which the polymerizable group R responds to any one of the formulae (II), (III) or (IV) below:

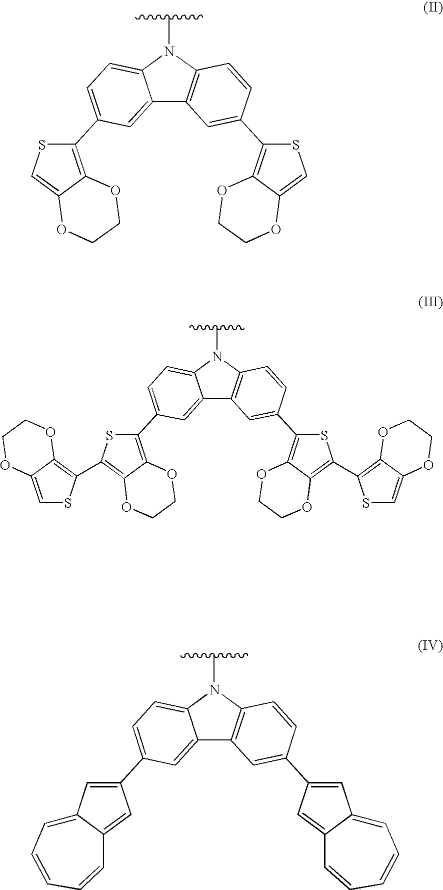

18. Compound according to claim 10, which responds to the formula (VI) below:

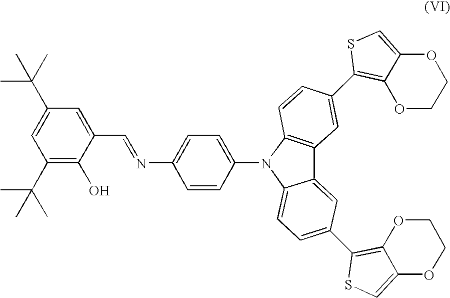

19. Polymer material, which is obtained by polymerization of a compound as defined in any one of claims 1 to 18.

20. Polymer material according to claim 19, which is in the form of a film.

21. Polymer material according to claim 19, which is obtained by electropolymerization.
